(19) United States
(12) Patent Application Publication
(10) Pub. No.: US 2018/0045800 A1
(43) Pub. Date: Feb. 15, 2018

(54) SCAN GEOMETRY PLANNING METHOD FOR MRI OR CT

(71) Applicant: KONINKLIJKE PHILIPS N.V., EINDHOVEN (NL)

(72) Inventors: TIM NIELSEN, EINDHOVEN (NL); PETER KOKEN, EINDHOVEN (NL)

(21) Appl. No.: 15/550,804

(22) PCT Filed: Feb. 23, 2016
(86) PCT No.: PCT/EP2016/053727

§ 371 Date: Aug. 14, 2017

(30) Foreign Application Priority Data
Feb. 24, 2015 (EP) 15156238.6

(51) Int. Cl. G01R 33/54; A61B 6/03; A61B 6/00; A61B 5/055

(52) U.S. Cl. CPC G01R 33/543; G01R 33/546; A61B 5/055; A61B 6/032; A61B 6/545; A61B 6/563; A61B 6/566; A61B 6/582

(57) ABSTRACT

The present invention relates to a method for scan geometry planning. The method comprises: providing at least one scanning imaging system (203A-N, 100) coupled to a computer server (201); controlling the server (201) to communicate with the at least one scanning imaging system (203A-N); building a database (400) from image data obtained from the at least one scanning imaging system, wherein the image data indicates scan 5 geometries in association with respective reference images acquired using the scan geometries; receiving at the server (201) a scan geometry request from a scanning imaging system (203A) of the at least one scanning imaging system, the request being indicative of a survey image, wherein the survey image is obtained during a calibration scan by imaging a patient by the requesting scanning imaging system (203A); comparing the survey image with 10 the reference images; sending the requesting scanning imaging system data indicative of a scan geometry associated with a reference image of the reference images that matches the survey image; controlling the requesting scanning imaging system (203A) to acquire imaging data during a clinical scan of the body volume using one of the submitted scan geometry and a modified scan geometry that results from the modification of the submitted scan geometry 15 at the requesting scanning imaging system; controlling the requesting scanning imaging system (203A) to send the modified scan geometry to the server (201) in case the modified scan geometry is used for the acquiring of imaging data.

### TECHNICAL FIELD OF THE INVENTION

The invention relates to scanning imaging systems, in particular to a method of scan geometry planning.

### BACKGROUND OF THE INVENTION

Automation and simplification of scanning imaging systems such as magnetic resonance imaging (MRI) systems is currently in the focus of research. One important feature that enables the fully automated magnetic resonance acquisition is automated planning of scan geometries.

The extension of automated scan planning methods to different anatomies and organs requires the development of anatomical models together with models for an expected image contrast for each anatomy and acquisition protocol. This is a complicated and also labour intensive development and research task and needs a huge amount of resources.

U.S. Pat. No. 8,144,955 concerns the automatic computation of a geometry plan from input landmark details. The US-patent application US2002/0198447 concerns a prescription of scanning parameters (scan geometry). This known method compares the current positioning of the patient to be examined to the positioning during a previous examination.

### SUMMARY OF THE INVENTION

Various embodiments provide for a method of scan geometry planning, a non-transitory computer-readable medium, a scanning imaging system and a network of scanning imaging systems as described by the subject matter of the independent claims. Advantageous embodiments are described in the dependent claims.

In one aspect, the invention relates to a method for scan geometry planning. The method comprises: providing at least one scanning imaging system coupled to a computer server; controlling the server (also referred to as the computer server) to communicate with the at least one scanning imaging system; building a database from image data obtained from the at least one scanning imaging system, wherein the image data indicates scan geometries in association with respective reference images acquired using the scan geometries; receiving at the server a scan geometry request from a scanning imaging system of the at least one scanning imaging system, the request being indicative of a survey image, wherein the survey image is obtained during a calibration scan by imaging a body volume of a patient by the requesting scanning imaging system; comparing the survey image with the reference images; sending the requesting scanning imaging system data indicative of a scan geometry associated with a reference image of the reference images that matches the survey image; controlling the requesting scanning imaging system to acquire imaging data during a clinical scan of the body volume using one of the submitted scan geometry and a modified scan geometry that results from the modification of the submitted scan geometry at the requesting scanning imaging system; controlling the requesting scanning imaging system to send the modified scan geometry to the server in case the modified scan geometry is used for the acquiring of imaging data. The requesting scanning imaging system is the scanning imaging system that sent the server the scan geometry request.

For example, the reference images may represent a same or different types of anatomical structures. Each reference image may at least show the anatomical structure that was of interest when the reference image was acquired. The anatomical structures may automatically be identified in the reference images using, for example, an anatomical model of these structures and the expected contrast.

The scan geometry that is associated with each of the reference images may be drawn into the reference image, e.g. by using a unique color that is reserved for scan geometry definition objects. The scan geometry may alternatively be tagged to the reference image or stored in a separate file or data set.

The above features may have the advantage of providing a centralized and automatic approach for scan geometry planning. This may increase the accuracy of the acquired image data at the scanning imaging systems since they are based on scan geometries obtained from a large sample of image data obtained from multiple scanning imaging systems.

Another advantage may be that the processing resources may be saved at the scanning imaging systems since the scan geometry planning process is performed centrally at the computer server.

The above features may further have the advantage of enabling a uniform acquisition of image data across multiple scanning imaging systems using a centralized scan geometry planning method.

The term “physical scan”, “clinical scan” or “main scan” refers to a scan for imaging an intended diagnosis image such as a T1 Weighted image, and it does not include a scan for acquiring MR signals for a calibration scan. The clinical scan is performed with a higher image resolution than the calibration scan.

The term “calibration scan” or “pre-scan” refers to a scan for determining imaging conditions and/or data used for image reconstruction etc. and it is performed separately from the clinical or main scan. The calibration scan may be performed before the clinical scan.

The term “scan geometry” refers to positional information that describe a target volume or an anatomical structure of the patient.

As used herein the term “server” or “computer server” refers to any computerized component, system or entity regardless of form that is adapted to provide data, files, applications, content, or other services to one or more other devices or entities.

According to one embodiment, building the database is performed during a pre defined time interval, the method further comprising controlling each of the at least one scanning imaging system to send at least part of the image data for successful scans only during at least part of the time interval.

A successful scan is a scan whose acquired image data correspond to expected results and/or fulfil predefined standard data acquisition norms.

The predetermined time interval is in fact the building time interval during which the database is built. Depending of the rate at which image data are received (uploaded to the server) this building time may be a few days, weeks, months, or even a year or a few years. During this building time interval, there is the at least part of the predetermined time interval, during which image data for successful scan only are sent (uploaded) to the server; this at least part thus can be indicated as a success time interval. The success time interval may be initially seet by the user. This embodiment may be advantageous as it may ease the workflow and may make efficient use of earlier successful geometry plans.

According to one embodiment, the method further comprises monitoring the amount of received image data; determining at a given point in time that the amount of received image data is higher than a predefined minimum sample size; dynamically determining the at least part of the time interval using the point in time. For example, the at least part of the time interval may have a starting time which is the time at which the building of the database starts and an end time that is the point in time. The minimum sample size may be defined so as to have a sample corresponding to successful scans large enough to increase the probability to find a scan geometry for a request of a scan geometry in that sample. Another advantage may be that the scanning imaging systems may not be constrained to send only data corresponding to successful scans but also data corresponding to unsuccessful scans, which may increase the sample used to select the scan geometry (although a stored scan geometry corresponds to a an unsuccessful scan at a given scanning imaging system it may still be usable for other scanning imaging systems and may generate successful scans).

That is, at one or more given points in time during building-up of the data base, i.e. during the building time, the amount of data in the database is determined. The amount of data may already be sufficient before the end of the set success time interval to have a sufficient likelihood that a scan geometry for a future geometry request can be found from the samples in the database. In this event, the remainder of the set success time interval may be employed to continue to send further image data (i.e. of more successful scans, but also of (allegedly) unsuccessful scans) to the server. In the event that, even in the set success time interval, the amount of data is considered insufficient to have a sufficient likelihood that future geometry requests can be met, then the success time interval may be extended until the amount of data is sufficient in that it exceeds the predefined minimum sample size. Accordingly, within the building time of the database, the amount of received image data is the dominant criterion on which continued database build-up is determined.

According to one embodiment, the method further comprises a method selected from the group consisting of: performing steps of building and receiving the request in parallel; performing the receiving step after the building step; performing the steps of building and receiving the request in parallel after the at least part of the first time interval is elapsed. This embodiment may be advantageous as it may provide a balance between the accuracy of the scan geometry planning (that dependents on the size of the amount of data built in the database) and processing time required to perform a scan geometry planning.

According to one embodiment, the method further comprises establishing a network of scanning imaging systems between the server and the at least one scanning imaging system; controlling the server and the at least one scanning imaging system to operate in a master-slave configuration in which the server is a master node and each of the at least one scanning imaging system is a slave node of the established network. This may facilitate the communication between the at least one scanning imaging system and the computer server by for example using a common communication protocol for exchanging data.

According to one embodiment, the image data further comprises for each scan geometry meta-data indicating the clinical scan, wherein the comparison is performed using the meta-data. For example, the meta-data may comprise an indication of a lesion and/or an anatomical structure. The present embodiment may enable an automatic offer of suitable geometry plan upon indication of the potential lesion.

According to one embodiment, the reference images stored in the database comprise 2D survey images obtained during calibration scans at the at least one scanning imaging system. This may have the advantage of speeding up the acquisition process compared to 3D survey images.

In another aspect, the invention relates to a non-transitory computer-readable medium, with instructions stored thereon, which when executed by at least one processor of a computing device, cause the computing device to perform the method steps of preceding claims.

In another aspect, the invention relates to a scanning imaging system. The scanning imaging system is configured to:
send image data to a computer server, wherein the image data indicates scan geometries in association with respective reference images acquired using the scan geometries at the scanning imaging system;
send a scan geometry request to the computer server, the request being indicative of a survey image, wherein the survey image is obtained during a calibration scan by imaging a body volume of a patient by the scanning imaging system;
receive data indicative of a scan geometry from the computer server;
acquire imaging data during a clinical scan of the body volume using one of the submitted scan geometry and a modified scan geometry that results from the modification of the submitted scan geometry by the scanning imaging system;
send the modified scan geometry to the computer server in case the modified scan geometry is used for the acquiring of imaging data.
   In another aspect, the invention relates to a network of scanning systems comprising at least one scanning imaging system and the computer server according to previous embodiments.

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example a data may be retrieved over a modem, over the internet, or over a local area network. Computer executable code embodied on a computer readable medium may be transmitted using any appropriate medium, including but not limited to wireless, wireline, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a processor. ‘Computer storage’ or ‘storage’ is a further example of a computer-readable storage medium. Computer storage is any non-volatile computer-readable storage medium. In some embodiments computer storage may also be computer memory or vice versa.

A ‘user interface’ as used herein is an interface which allows a user or operator to interact with a computer or computer system. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, gear sticks, steering wheel, pedals, wired glove, dance pad, remote control, and accelerometer are all examples of user interface components which enable the receiving of information or data from an operator.

A ‘hardware interface’ as used herein encompasses an interface which enables the processor of a computer system to interact with and/or control an external computing device and/or apparatus. A hardware interface may allow a processor to send control signals or instructions to an external computing device and/or apparatus. A hardware interface may also enable a processor to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

A ‘processor’ as used herein encompasses an electronic component which is able to execute a program or machine executable instruction. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. Many programs have their instructions performed by multiple processors that may be within the same computing device or which may even be distributed across multiple computing devices.

Magnetic resonance image data is defined herein as being the recorded measurements of radio frequency signals emitted by the subject's/object's atomic spins by the antenna of a Magnetic resonance apparatus during a magnetic resonance imaging scan. A Magnetic Resonance Imaging (MRI) image is defined herein as being the reconstructed two or three dimensional visualization of anatomic data contained within the magnetic resonance imaging data. This visualization can be performed using a computer.

It is understood that one or more of the aforementioned embodiments of the invention may be combined as long as the combined embodiments are not mutually exclusive.

### BRIEF DESCRIPTION OF THE DRAWINGS

In the following preferred embodiments of the invention will be described, by way of example only, and with reference to the drawings in which:

FIG. 1 illustrates a magnetic resonance imaging system,

FIG. 2 illustrates a system of scanning imaging systems,

FIG. 3 is a flowchart of a method for scan geometry planning,

FIG. 4 illustrates a structure of a data table according to the present disclosure.

### DETAILED DESCRIPTION OF THE EMBODIMENTS

In the following, like numbered elements in the figures are either similar elements or perform an equivalent function. Elements which have been discussed previously will not necessarily be discussed in later figures if the function is equivalent.

Various structures, systems and devices are schematically depicted in the figures for purposes of explanation only and so as to not obscure the present invention with details that are well known to those skilled in the art. Nevertheless, the attached figures are included to describe and explain illustrative examples of the disclosed subject matter.

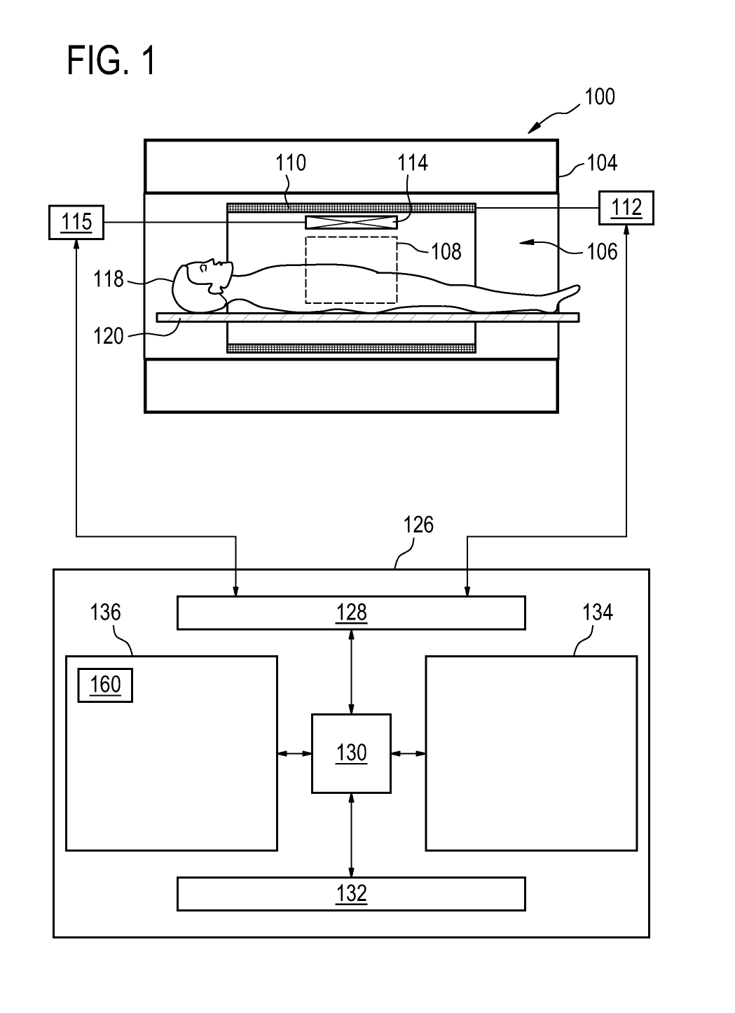

FIG. 1 illustrates an example scanning imaging system being a magnetic resonance imaging system 100. The magnetic resonance imaging system 100 comprises a magnet 104. The magnet 104 is a superconducting cylindrical type magnet 100 with a bore 106 through it. The use of different types of magnets is also possible for instance it is also possible to use both a split cylindrical magnet and a so called open magnet. A split cylindrical magnet is similar to a standard cylindrical magnet, except that the cryostat has been split into two sections to allow access to the iso-plane of the magnet, such magnets may for instance be used in conjunction with charged particle beam therapy. An open magnet has two magnet sections, one above the other with a space in-between that is large enough to receive a subject 118, the arrangement of the two sections area similar to that of a Helmholtz coil. Open magnets are popular, because the subject is less confined. Inside the cryostat of the cylindrical magnet there is a collection of superconducting coils. Within the bore 106 of the cylindrical magnet 104 there is an imaging zone 108 where the magnetic field is strong and uniform enough to perform magnetic resonance imaging.

Within the bore 106 of the magnet there is also a set of magnetic field gradient coils 110 which is used for acquisition of magnetic resonance data to spatially encode magnetic spins of a target volume within the imaging zone 108 of the magnet 104. The magnetic field gradient coils 110 are connected to a magnetic field gradient coil power supply 112. The magnetic field gradient coils 110 are intended to be representative. Typically magnetic field gradient coils 110 contain three separate sets of coils for spatially encoding in three orthogonal spatial directions. A magnetic field gradient power supply supplies current to the magnetic field gradient coils. The current supplied to the magnetic field gradient coils 110 is controlled as a function of time and may be ramped or pulsed.

MRI system 100 further comprises an RF transmit coil 114 above the subject 118 and adjacent to the imaging zone 108 for generating RF excitation pulses. The RF transmit coil 114 may include for example a set of surface coils or other specialized RF coils. The RF transmit coil 114 may be used alternately for transmission of RF pulses as well as for reception of magnetic resonance signals e.g., the RF transmit coil 114 may be implemented as a transmit array coil comprising a plurality of RF transmit coils. The RF transmit coil 114 is connected to an RF amplifier 115.

The magnetic field gradient coil power supply 112 and the RF amplifier 115 are connected to a hardware interface 128 of computer system 126. The computer system 126 further comprises a processor 130. The processor 130 is connected to the hardware interface 128, a user interface 132, a computer storage 134, and computer memory 136.

The computer memory 136 is shown as containing a control module 160. The control module 160 contains computer-executable code which enables the processor 130 to control the operation and function of the magnetic resonance imaging system 100. It also enables the basic operations of the magnetic resonance imaging system 100 such as the acquisition of magnetic resonance data.

The MRI system 100 may be configured to acquire imaging data from the patient 118 in calibration and/or physical scans. For example, the MRI system 100 may be configured to first acquire first imaging data using a calibration scan (or pilot scan), and after that a physical scan may be performed to acquire second imaging data by for example using the outcome e.g. first imaging data of the calibration scan.

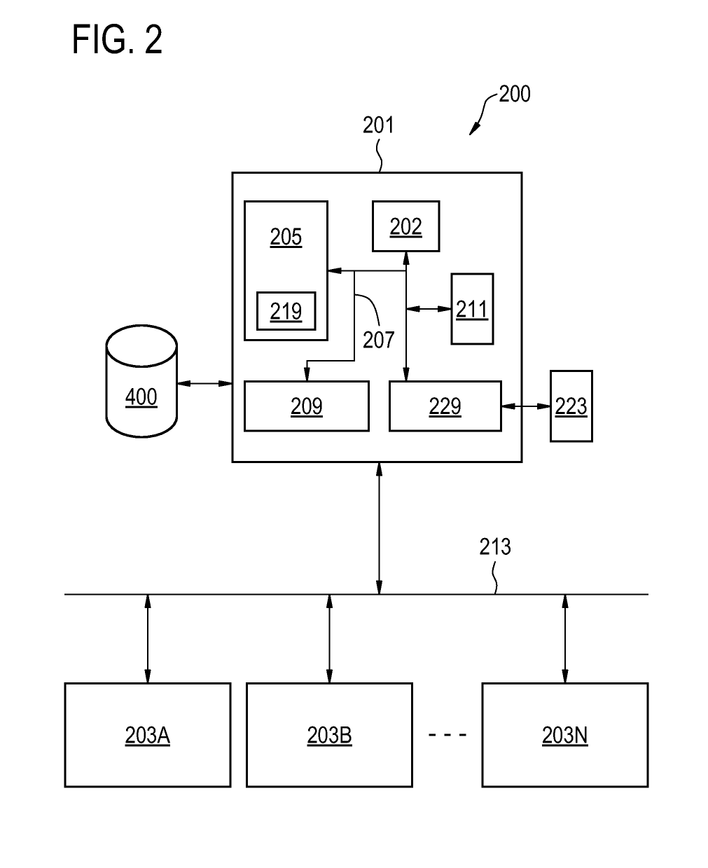

FIG. 2 depicts an exemplary architecture of a medical system 200. The medical system 200 comprises a computer serer 201. The computer server 201 is in communication with one or more scanning imaging systems 203A-N via network 213. The network 213 may comprise a local area network (LAN), a general wide area network (WAN), and/or a public network (e.g., the Internet). A scanning imaging system of the one or more scanning imaging systems 203A-N may comprise a MRI imaging system as described with reference to FIG. 1 or a computed tomography (CT) system.

The components of computer server 201 may include, but are not limited to, one or more processors or processing units 202, a storage system 211, a memory system 205, and a bus 207 that couples various system components including memory system 105 to processor 202. Memory system 205 may include computer system readable media in the form of volatile memory, such as random access memory (RAM) and/or cache memory.

Computer server 201 typically includes a variety of computer system readable media. Such media may be any available media that is accessible by computer server 201, and it includes both volatile and non-volatile media, removable and non-removable media.

Computer server 201 may also communicate with one or more external devices such as a keyboard, a pointing device, a display 223, etc.; one or more devices that enable a user to interact with computer server 201; and/or any devices (e.g., network card, modem, etc.) that enable computer server 201 to communicate with one or more other devices such as the scanning imagine systems 203A-N. Such communication can occur via I/O interface(s) 229. Still yet, computer server 201 may communicate with the network 213 via network adapter 209. As depicted, network adapter 209 communicates with the other components of computer server 201 via bus 207.

Memory system 205 is configured to store a control unit 219. The control unit 219 may be configured to receive from the one or more scanning imaging systems 203A-N image data. The image data may indicate, for example, scan geometries that have been used by a scanning imaging system in order to acquire data e.g. MRI data in a clinical scan. In another example, the image data may indicate scan geometries that have been used by a scanning imaging system in order to acquire data e.g. MRI data in a pilot or calibration scan.

For example, MRI system 203A may be controlled to acquire imaging data from a patient e.g. 118. For that, the MRI system 203A may, for example, define a number of scan geometries for acquiring the imaging data from at least one region of interest relative to the patient and may perform at least one scan for acquiring the imaging data in accordance with at least one of the defined scan geometries. After acquiring the imaging data, the MRI system 203A may send image data to the computer server 201; the image data is indicative of the at least one scan geometry and the acquired imaging data (e.g. in the form of reconstructed MRI images i.e. reference images) that may be used by the computer server 201 as reference images. The image data may further indicate, for example, age, weight, size of the patient as well as diagnostic questions/previous acquired findings from other imaging modalities or any kind of information from the patient's history that may be used for selecting the scan geometry. For example, if a new exam on a patient is performed by a scanning imaging system, the scanning imaging system transmits survey images to the computer server. Also, the image data may comprise meta-information and the scan protocol of the clinical scan for which the scan geometry should be planned. However, the scanning imaging system is configured to not send personal patient data to avoid privacy protection issues. On the computer server, a current survey image is compared to other received survey images with the same clinical question and the best match is identified. Simple quality assessment can be performed on the survey images. If the quality is under a given threshold (e.g. strong breathing motion artefacts) reacquiring can be requested to a user of the scanning imaging system.

The received image data e.g. in the form or reconstructed MRI images may be used as reference images for subsequent scans. The reference images may be combined or represented through the use of one or more atlases; the one or more atlases may be compared with received e.g. survey images in order to select a scan geometry. For example, a statistical atlas representative of the received image data may be acquired. The statistical atlas may be used for comparison with other acquired images. For example, scan geometries may be linked for different anatomical structures in order to build and map an anatomical atlas.

The received image data may comprise 2D survey images (or reference images) obtained during calibration scans at the at least one scanning imaging system 203A-N.

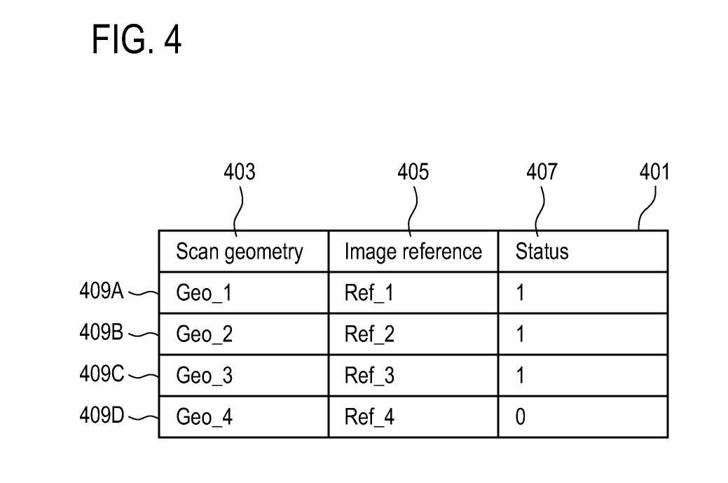

Using the received image data from the one or more scanning imaging systems 203A-N, the control unit 219 may, for example, build a database 400. The database 400 may comprise reference images and associated scan geometries. An example structure of the data stored in the database 400 is shown in FIG. 4.

In one embodiment, the building of the database 400 (e.g. storing data in the database 400) may be performed during a predefined first time interval only with successful scans. That is, the control unit 219 may control each of the at least one scanning imaging system 203A-N to send the image data for successful scans only during the first time interval. In another example, the control unit 219 may control the at least one scanning imaging system 203A-N to send a status information in association with the image data to indicate whether the scan that produced image references in the image data is successful or not. The control unit 219 may use the value of the status information to accept or reject the received image data during the first time interval. During a further following second time interval the control unit 219 may not select the image data using the status information i.e. may accept all the image data received including the image data of unsuccessful scans. This may provide an initial sample for which the size may be controlled (e.g. by changing the first time interval) to include data from successful scans.

In one embodiment, the network 213 may be established between the computer server 201 and the at least one scanning imaging system 203A-N. For example, the network 213 may be a local area network wherein the scanning imaging systems 203A-N belong to a single building such as a hospital. In another example, the network 213 may be a wide area network that provides communication services in a geographic area larger than that served by a local area network.

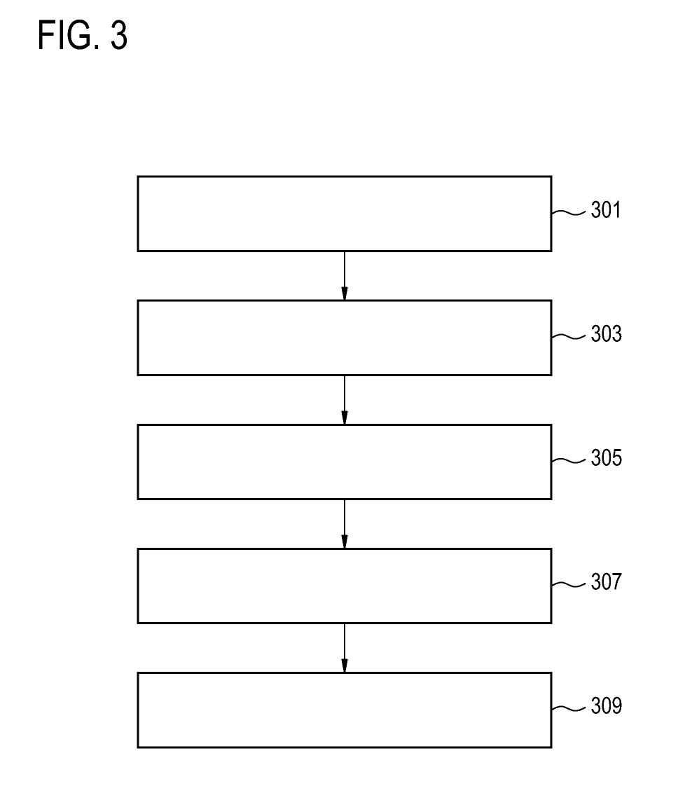

The computer server 201 and the at least one scanning imaging system 203A-N may be controlled to operate in a master-slave configuration in which the computer server 201 is a master node and each of the at least one scanning imaging system 203A-N is a slave node of the established network 213. This may enable a unidirectional control by the server 201 over the scanning imaging systems 203A-N. The operation of the computer server 201 as well as the at least one scanning imaging system 203A-N will be described in details with reference to FIG. 3.

FIG. 3 is a flowchart of a method for scan geometry planning In step 301, the control unit 219 may receive a scan geometry request from a scanning imaging system e.g. 203A of the at least one scanning imaging system 203A-N. The request is indicative of a survey image, wherein the survey image is obtained during a calibration scan by imaging e.g. a body volume of a patient e.g. 118 by the requesting scanning imaging system 203A. Step 301 of receiving the scan geometry request may be performed in parallel to building the database 400 (as described above).

In another example, step 301 may be performed after the building of the database 400 is finished.

In a further example, step 301 may be performed in parallel to the building of the database 400 after the first time interval is elapsed. Having the initial sample big enough may increase the probability of finding a scan geometry of a successful scan that satisfies the scan geometry request.

In step 303, the control unit 219 may compare the survey image with the reference images that are stored in the database. The comparison may be performed for example by comparing each pixel or voxel of the survey image to a corresponding pixel or voxel of the reference images or the combined images using the one or more atlases. The comparison may be performed by for example performing a registration between the atlases/reference images and the survey image.

In step 305, the control unit 219 may send the requesting scanning imaging system 203A data indicative of a scan geometry associated with a reference image of the reference images that matches the survey image. The matching reference image may be selected by identifying that pixels or voxels, or groups of pixels or voxels, of the survey image for which the measure of difference to the selected reference image is smaller than a preset threshold. For example, the selection may be performed using (described above) meta-data of the received image data.

The control unit 219 may control in step 307 the requesting scanning imaging system 203 A (e.g. by sending a control signal) to acquire imaging data during a clinical scan of the body volume using one of the submitted scan geometry and a modified scan geometry that results from the modification of the submitted scan geometry at the requesting scanning imaging system. For example, the scanning imaging system 203A may receive from a user of the scanning imaging system 203A a validation of parameters of the received scan geometry and thus the scanning imaging system may use the received scan geometry without modification in order to perform the clinical scan. In another example, the requesting scanning imaging system 203A may be allowed or configured to adapt or modify the submitted scan geometry for performing the clinical scan. The adaptation may for example take into account user's adjustments e.g. using user inputs/adjustments in order to adapt the submitted scan geometry.

In step 309, the control unit 219 may control the requesting scanning imaging system 203A to send the modified scan geometry to the computer server 201 in case the modified scan geometry is used for the acquiring of imaging data. For example, the modified scan geometry may be used to replace the submitted scan geometry in the database.

For example, an advantage of the database is to match input data (current survey, requested scan protocol and clinical question . . . ) to the most similar scan stored in the database. This may, for example, be achieved by clustering techniques known from big data analytics. In another example, the analyzing of the data received at the server 201, several sub-groups of “scan geometry planning traditions” can be identified which allows making a differentiated proposal taking local customs into account (e.g. differences between US and Europe or different hospitals). Central quality control could be implemented by having these groups of “scan geometry planning traditions” reviewed by radiologists who rank them according to appropriateness. For example, the reference images may be combined or represented through the use of one or more atlases based on properties of the scanning imaging systems 203A-N. The properties may comprise location, type/model etc of the scanning imaging systems. For example, reference images received from scanning imaging systems located in Europe may be combined in one or more atlases, while reference images received from scanning imaging systems located in US may be combined in one or more other atlases.

FIG. 4 shows an example data structure of data stored in the database 400. However; a skilled person having access to the present disclosure would understand that other data structures may be used. The data structure may comprise for example a data table 401. The field 403 of the data table 401 may comprise information on a given scan geometry Geo_1-4. The information e.g. Geo_1 of the given scan geometry may comprise data descriptive of the given scan geometry and/or a link or a reference to another data source such as a text file descriptive of the given scan geometry. The field 405 of the data table 401 may comprise an indication e.g. Ref_1 of a reference image (e.g. a link to where the reference image is stored). The field 407 may be an optional field that comprises a status information indicative of the status of the scan that has been used to acquire the reference image. The status information may have for example two values (0 or 1) indicative of successful and unsuccessful scans. Each row 409A-D of the data table 401 may further indicate the scanning imaging system 203A-N that provided the data stored in that row. For example, data row 409A may comprise data related to scanning imaging system 203A. Data row 409B may comprise data related to scanning imaging system 203B etc.

### LIST OF REFERENCE NUMERALS

100 magnetic resonance imaging system
104 magnet
106 bore of magnet
108 imaging zone
110 magnetic field gradient coils
112 magnetic field gradient coil power supply
114 radio-frequency coil
115 RF amplifier
118 subject
126 computer system
128 hardware interface
130 processor
132 user interface
134 computer storage
136 computer memory
160 control module
201 computer server
202 processor
203A scanning imaging system
203B scanning imaging system
203N scanning imaging system
205 memory
207 bus
209 network adapter
211 storage
213 network
219 control unit
223 external device
229 I/O interface
400 database
301-309 steps
401 data table
403-405 fields
409A-D rows.

1. A method for scan geometry planning comprising:
providing at least one scanning imaging system coupled to a computer server;
controlling the server to communicate with the at least one scanning imaging system;
building a database from image data obtained from the at least one scanning imaging system, wherein the image data indicates scan geometries in association with respective reference images acquired using the scan geometries;
receiving at the server a scan geometry request from a scanning imaging system, of the at least one scanning imaging system, the request being indicative of a survey image, wherein the survey image is obtained during a calibration scan by imaging a patient by the requesting scanning imaging system;
comparing the survey image with the reference images;
sending the requesting scanning imaging system data indicative of a scan geometry associated with a reference image of the reference images that matches the survey image;
controlling the requesting scanning imaging system to acquire imaging data during a clinical scan of the patient using one of the submitted scan geometry and a modified scan geometry that results from the modification of the submitted scan geometry at the requesting scanning imaging system;
controlling the requesting scanning imaging system to send the modified scan geometry to the server in case the modified scan geometry is used for the acquiring of imaging data and, wherein
building the database is performed during a pre defined time interval, the method further comprising controlling each of the at least one scanning imaging system(s) to send at least part of the image data for successful scans only during at least part of the time interval.

2. The method of claim 1, further comprising:
monitoring the amount of received image data;
determining at a given point in time that the amount of received image data is higher than a predefined minimum sample size;
dynamically determining the at least part of the time interval using the point in time.

3. The method of claim 1, further comprising a method selected from the group consisting of:
performing steps of building and receiving the request in parallel;
performing the receiving step after the building step;
performing the steps of building and receiving the request in parallel after the at least part of the first time interval is elapsed.

4. The method of claim 1, further comprising:
establishing a network of scanning imaging systems between the server and the at least one scanning imaging system,
controlling the server and the at least one scanning imaging system to operate in a master-slave configuration in which the server is a master node and each of the at least one scanning imaging system is a slave node of the established network.

5. The method of claim 1, wherein the image data further comprises for each scan geometry meta-data indicating the clinical scan, wherein the comparison is performed using the meta-data.

6. The method of claim 1, wherein the reference images stored in the database comprise 2D survey images obtained during calibration scans at the at least one scanning imaging system.

7. A non-transitory computer-readable medium, with instructions stored thereon, which when executed by at least one processor of a computing device, cause the computing device to perform the method steps of claim 1.

8. A scanning imaging system being configured to:
send image data to a computer server, to build-up a database from the image data wherein the image data indicates scan geometries in association with respective reference images acquired using the scan geometries at the scanning imaging system; and sending the image data to build-up the database is performed during a pre defined time interval
control the scanning imaging system to send at least part of the image data for successful scans only during at least part of the time interval,
send a scan geometry request to the computer server, the request being indicative of a survey image, wherein the survey image is obtained during a calibration scan by imaging a body volume of a patient by the scanning imaging system;
receive data indicative of a scan geometry from the computer server;
acquire imaging data during a clinical scan of the body volume using one of the submitted scan geometry and a modified scan geometry that results from the modification of the submitted scan geometry by the scanning imaging system;
send the modified scan geometry to the computer server in case the modified scan geometry is used for the acquiring of imaging data,

9. A network of scanning systems comprising at least one scanning imaging system of claim 9 and the computer server.

10. A method for building a database at a server of scan geometry planning the method comprising:
building the database from image data obtained from at least one scanning imaging system, wherein the image data indicates scan geometries in association with respective reference images acquired using the scan geometries;
receiving at the server a scan geometry request from a scanning imaging system of the at least one scanning imaging system, the request being indicative of a survey image, wherein the survey image is obtained during a calibration scan by imaging a patient by the requesting scanning imaging system;
comparing the survey image with the reference images;
sending the requesting scanning imaging system data indicative of a scan geometry associated with a reference image of the reference images that matches the survey image;
controlling the requesting scanning imaging system to acquire imaging data during a clinical scan of the patient using one of the submitted scan geometry and a modified scan geometry that results from the modification of the submitted scan geometry at the requesting scanning imaging system;
controlling the requesting scanning imaging system to send the modified scan geometry to the server in case the modified scan geometry is used for the acquiring of imaging data and, wherein
building the database is performed during a pre defined time interval, the method further comprising controlling each of the at least one scanning imaging system to send at least part of the image data for successful scans only during at least part of the time interval.

11. A method as claimed in claim 11, comprising the steps of
monitoring the amount of received image data;
determining at a given point in time that the amount of received image data is higher than a predefined minimum sample size;
dynamically determining the at least part of the time interval using the point in time.
